SEQUENCE LISTING (1 sequence)

SEQ ID NO: 1 - protein, 201 aa, Homo sapiens [sequence omitted]

(19) United States
(12) Patent Application Publication
(10) Pub. No.: US 2006/0105419 A1
(43) Pub. Date: May 18, 2006

(54) Use of a glutathione peroxidase 1 as a marker in cardiovascular conditions

(75) Inventors: Stefan Blankenberg, Kriftel (DE); Karl J. Lackner, Mainz (DE); Hans J. Rupprecht, Mainz (DE); Christoph Bickel, Koblenz (DE)

Correspondence Address: FOLEY & LARDNER LLP, P.O. BOX 80278, SAN DIEGO, CA, 92138-0278, US

(73) Assignee: Biosite, Inc.

(21) Appl. No.: 11/204,764

(22) Filed: Aug. 15, 2005

Related U.S. Application Data
(60) Provisional application No. 60/602,997, filed on Aug. 16, 2004.

(51) Int. Cl. C12Q 1/26

(52) U.S. Cl. 435/25

(57) ABSTRACT

The present invention relates to materials and procedures for evaluating patients suffering from cardiovascular conditions, particularly acute coronary syndromes. In particular, the presence, amount, or enzymatic activity of glutathione peroxidase-1 in a patient sample, alone or in combination with one or more other markers, provides diagnostic and/or prognostic information. While applicable to diseases and conditions in which inflammation is generally manifested, the methods and compositions described herein are particularly applicable to acute coronary syndromes, including conditions selected from the group consisting of unstable angina, non-ST-elevation non-Q wave myocardial infarction, ST-elevation non-Q wave MI, and transmural (Q-wave) MI.

### CROSS-REFERENCE TO RELATED PATENT APPLICATIONS

This application claims the benefit under 35 USC § 19(e) of U.S. Application Ser. No. 60/602,997, filed Aug. 16, 2004.

### FIELD OF THE INVENTION

The present invention relates in part to methods, compositions, and devices for the measurement of glutathione peroxidase 1, and the use of such measurement in the diagnosis, prognosis, and treatment of patients with cardiovascular conditions.

### BACKGROUND OF THE INVENTION

The following discussion of the background of the invention is merely provided to aid the reader in understanding the invention and is not admitted to describe or constitute prior art to the present invention.

The term “cardiovascular conditions” refers to a diverse set of disorders of the heart and vasculature, including atherosclerosis, ischemic stroke, intracerebral hemorrhage, subarachnoid hemorrhage, transient ischemic attack, systolic dysfunction, diastolic dysfunction, aneurysm, aortic dissection, myocardial ischemia, angina pectoris, myocardial infarction, congestive heart failure, dilated congestive cardiomyopathy, hypertrophic cardiomyopathy, restrictive cardiomyopathy, cor pulmonale, arrhythmia, valvular heart disease, endocarditis, pulmonary embolism, venous thrombosis, peripheral vascular disease, and acute coronary syndromes. Major cardiovascular conditions may present with few overt symptoms, such as pain, dyspnea, weakness, palpitations, and dizziness. The clinical presentation of these various conditions can often be strikingly similar, even though the underlying disease, and the appropriate treatments to be given to one suffering from the various diseases, can be completely distinct.

Workers seeking to provide rapid diagnostic (that is, the presence of a particular condition or disease) and/or prognostic (that is, a prediction of some future outcome) information for various cardiovascular diseases or conditions have sought to identify subject-derived “markers” that are indicative of a particular diagnosis or prognosis. In the case of a “diagnostic marker,” these are molecules that are preferably present in a sample obtained from a first subject suffering from a condition or disease in an amount that differs (either a greater or lesser amount) from the amount present in a sample from a second “normal” subject. In the case of a “prognostic marker,” these are molecules that are preferably present in a sample obtained from a first subject predisposed to some future outcome in an amount that differs from the amount present in a sample from a second subject (e.g., a subject suffering from the same condition as the first subject, a subject suffering from a different condition, or a normal subject).

For example, epidemiological studies have shown an association between circulating levels of certain inflammatory markers and coronary artery disease (CAD). C-reactive protein (“CRP”) has been reported to be predictive for future coronary events. See, e.g., Kuller et al., Am. J. Epidemiol. 144:537-47, 1996; Haverkate et al., Lancet 349:462-66, 1997; Ridker et al., N. Engl. J. Med. 342:836-43, 2000. Other markers, including cardiac-specific troponin, CK-MB, myoglobin, interleukin (“IL”)-6, soluble adhesion molecules, IL-18, and tumor necrosis factor-α (“TNF-α”) have also been reported to be potential tools for cardiovascular diagnosis and/or risk prediction. See, e.g., Ridker et al., Circulation 101:1767-72, 2000; Volpato et al., Circulation 103:947-53, 2001; Ridker et al., Lancet 351:88-92, 1998; Ridker Circulation 103:491-95, 2001; Barbaux et al., Arterioscler. Thromb. Vasc. Biol. 21:1668-73, 2001; Blankenberg et al., Circulation 104:1336-42,2001; Blankenberg et al., Circulation 106:24-30, 2002; Koukkunen et al., Ann. Med. 33:37-47, 2001; Ridker et al., Circulation 101:2149-53, 2000; Rallidis et al., Heart 90:25-9, 2004.

In practice, the sensitivity and specificity of a marker for a particular diagnosis or prognosis is typically assessed using a “diseased” population and a “control” (e.g., a normal) population. While the terms “diseased” and “control” are used for convenience herein to refer to these populations, these terms refer to a first subject population exhibiting some characteristic of interest, and a second subject population not exhibiting that characteristic. That characteristic might be the presence or absence of a disease, a risk of some future outcome, etc. Receiver Operating Characteristic curves, or “ROC” curves, may be calculated by plotting the value of a variable versus its relative frequency in the “control” and “disease” populations. For any particular marker, a distribution of marker levels for subjects exhibiting and not exhibiting the characteristic of interest will likely overlap. Such a test need not absolutely distinguish “control” from “disease” with 100% accuracy, and the area of overlap indicates where the test cannot distinguish the control population from the disease population. A threshold value for the test is selected, above which (or below which, depending on how a marker changes with the disease) the test is considered to be indicative of one state or condition in a subject (e.g., disease, outcome, etc.) and below which the test is considered to be indicative of another state or condition in the subject. The area under the ROC curve is a measure of the probability that the perceived measurement will allow correct identification of a characteristic of interest. These methods are well known in the art. See, e.g., Hanley et al., Radiology 143: 29-36 (1982).

The acuteness or severity of the symptoms often dictates how rapidly a diagnosis must be established and treatment initiated. For example, immediate diagnosis and care of a patient experiencing a variety of acute conditions can be critical. See, e.g., Harris, Aust. Fam. Physician 31: 802-06 (2002) (asthma); Goldhaber, Eur. Respir. J. Suppl. 35: 22s-27s (2002) (pulmonary embolism); Lundergan et al., Am. Heart J. 144: 456-62 (2002) (myocardial infarction). However, even in cases where the apparent symptoms appear relatively stable, rapid diagnosis, and the rapid initiation of treatment, can provide both relief from immediate discomfort and advantageous improvement in prognosis.

There remains in the art the need to identify markers useful in evaluating patient diagnosis and prognosis within the spectrum of cardiovascular conditions, so that patients at risk of morbidity and/or death or can be identified and treated.

### SUMMARY OF THE INVENTION

The present invention relates to materials and procedures for diagnosing subjects suffering from one or more cardiovascular conditions or diseases, for evaluating the prognosis of subjects, and/or for treating subjects experiencing or predisposed to oxidative stress. In particular, the level of erythrocyte intracellular glutathione peroxidase-1 (“GPx-1”) activity in a subject sample, alone or in combination with one or more additional subject-derived markers, can provide diagnostic and/or prognostic information useful for predicting morbidity and/or mortality, particularly in subjects suffering from an acute coronary syndrome. In addition, levels of GPx-1 activity may be used to select a treatment regimen, and indicate subjects that may benefit from antioxidant therapy.

In various aspects, the invention relates to materials and procedures identifying levels of GPx-1 activity that are associated with an increased predisposition to a particular (either adverse or beneficial) outcome in a patient; for identifying levels of GPx-1 activity that are associated with a particular diagnosis; identifying one or more additional markers that increase the predictive value of a level of GPx-1 activity; using the level of GPx-1 activity in a subject sample, alone or in combination with one or more additional markers, to assign a diagnosis and/or prognosis to a subject; and using the GPx-1 level, alone or in combination with one or more additional markers, to determine and/or monitor a treatment regimen for a subject.

Thus, the materials and procedures described herein can be used to identify those patients that may be at increased risk for one or more serious complications, including the risk of death, resulting from one or more cardiovascular conditions, and to guide the clinician in treatment of such patients.

It is a first object of the invention to provide methods for determining the prognosis of a subject diagnosed with one or more cardiovascular conditions. In certain aspects, the present invention provides methods for identifying a level of GPx-1 activity that is associated with an increased predisposition of a particular outcome. In related aspects, the level of GPx-1 activity in a sample obtained from a subject can be measured, and compared to a prognostic level that is associated with an increased predisposition for a particular outcome. By correlating the subject level to the prognostic level, a prognosis can be assigned to the subject.

The term “intracellular glutathione peroxidase-1” or “GPx-1” (EC 1.11.1.9) as used herein refers to an enzyme that catalyzes the reduction of hydrogen peroxide and organic peroxides via the oxidation of glutathione, it also acts as a peroxynitrite reductase. GPx-1 contains an active site selenocysteine (residue 47) encoded by the opal UGA codon, which is otherwise one of the three translation termination codons. Certain variant forms of GPx-1 (containing, for example, a proline→leucine substitution at residue 198, an inserted alanine at residue 11, and/or a leucine→glutamine substitution at residue 91) are also known in the art. In addition, other forms of GPx-1 may be generated by covalent modification of a parent polypeptide, for example by oxidation of methionine residues, ubiquitination, cysteinylation, nitrosylation, glycosylation, etc. Moreover, additional forms of GPx-1 may be generated by proteolysis to generate additional forms of GPx-1. All such forms of GPx-1 are subjects of the present invention. Particularly preferred GPx-1 for use in the methods described herein may be obtained from erythrocyte cytosol as described hereinafter.

The phrase “determining the prognosis” as used herein refers to methods by which the skilled artisan can predict the course or outcome of a condition in a patient. The term “prognosis” does not refer to the ability to predict the course or outcome of a condition with 100% accuracy, or even that a given course or outcome is more likely to occur than not. Instead, the skilled artisan will understand that the term “prognosis” refers to an increased probability that a certain course or outcome will occur; that is, that a course or outcome is more likely to occur in a patient exhibiting a given characteristic, such as the presence or level of a prognostic indicator, when compared to those individuals not exhibiting the characteristic. In certain embodiments, GPx-1 may be correlated to a prognosis by merely its presence or absence. For example, an assay can be designed so that a positive signal for a marker only occurs above a particular threshold concentration of interest, and below which concentration the assay provides no signal above background. In other embodiments, threshold concentration of GPx-1 can be established, and the level of GPx-1 in a patient sample can simply be compared to the threshold level.

Often, a positive likelihood ratio, negative likelihood ratio, odds ratio, or hazard ratio is used as a measure of a test's ability to predict risk or diagnose a condition or disease. In the case of a positive likelihood ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In the case of a negative likelihood ratio, a value of 1 indicates that a negative result is equally likely among subjects in both the “diseased” and “control” groups; a value greater than 1 indicates that a negative result is more likely in the test group; and a value less than 1 indicates that a negative result is more likely in the control group. In certain preferred embodiments, one or more GPx-1 thresholds may be preferably selected to exhibit a positive or negative likelihood ratio of at least about 1.5 or more or about 0.67 or less, more preferably at least about 2 or more or about 0.5 or less, still more preferably at least about 5 or more or about 0.2 or less, even more preferably at least about 10 or more or about 0.1 or less, and most preferably at least about 20 or more or about 0.05 or less. The term “about” in this context refers to +/−5% of a given measurement.

In the case of an odds ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In certain preferred embodiments, one or more GPx-1 thresholds may be preferably selected to exhibit an odds ratio of at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less. The term “about” in this context refers to +/−5% of a given measurement.

In the case of a hazard ratio, a value of 1 indicates that the relative risk of an endpoint (e.g., death) is equal in both the “diseased” and “control” groups; a value greater than 1 indicates that the risk is greater in the diseased group; and a value less than 1 indicates that the risk is greater in the control group. In certain preferred embodiments, one or more GPx-1 thresholds may be preferably selected to exhibit a hazard ratio of at least about 1.1 or more or about 0.91 or less, more preferably at least about 1.25 or more or about 0.8 or less, still more preferably at least about 1.5 or more or about 0.67 or less, even more preferably at least about 2 or more or about 0.5 or less, and most preferably at least about 2.5 or more or about 0.4 or less. The term “about” in this context refers to +/−5% of a given measurement.

The skilled artisan will understand that associating a prognostic indicator with a predisposition to a particular outcome is a statistical analysis. Statistical significance is often determined by comparing two or more populations, and determining a confidence interval and/or a p value. See, e.g., Dowdy and Wearden, Statistics for Research, John Wiley & Sons, New York, 1983. Preferred confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001. Exemplary statistical tests for associating a prognostic indicator with a predisposition to an adverse outcome are described hereinafter.

The term “correlating,” as used herein in reference to the use of prognostic indicators to determine a prognosis, refers to comparing the presence or amount of the prognostic indicator in a subject sample to its presence or amount in subjects known to suffer from, or known to be at risk of, a given condition; or in subjects known to be free of a given condition. For example, a level of GPx-1 activity in a patient can be compared to a level known to be associated with an increased disposition for a future MI, future congestive heart failure, future stroke, future neurological impairment, future pulmonary injury, future renal injury, future stable angina, future unstable angina, future need for rehospitalization, future need for revascularization, or future death. The patient's level of GPx-1 activity is said to have been correlated with a prognosis; that is, the skilled artisan can use the patient's level of GPx-1 activity to determine the likelihood that the patient is at risk for one of these or other outcomes, and respond accordingly. Alternatively, the patient's level of GPx-1 activity can be compared to a level of GPx-1 activity known to be associated with a positive outcome (e.g., no MI, no death, etc.), and determine if the patient's prognosis is predisposed to the positive outcome.

In certain preferred embodiments, GPx-1 is applied to determine a prognosis in a subject suffering from an acute coronary syndrome, or in a clinically normal subject. The phrase “acute coronary syndromes” as used herein refers to a group of coronary disorders that result from ischemic and/or necrotic insult to the heart. ACS includes unstable angina, non-ST-elevation non-Q wave MI, ST-elevation non-Q wave MI, and transmural (Q-wave) MI. ACS can be divided into non-ST-elevation ACS and ST-elevation ACS, each of which may be associated with certain prognostic indicators and prognoses, as described herein. The phrase “non-ST-elevation acute coronary syndrome” refers to those ACS not associated with an elevated ST component in an electrocardiogram. Non-ST-elevation ACS include unstable angina and non-ST-elevation non-Q wave MI. See, e.g., Nyman et al., J. Intern. Med. 1993; 234: 293-301, 1993; Patel et al., Heart 75: 222-28, 1996; Patel et al., Eur. Heart J. 19: 240-49, 1998; and Lloyd-Jones et al., Am. J. Cardiol. 81: 1182-86, 1998.

Diagnosis of ACS generally, and non-ST-elevation ACS in particular, is well known to the skilled artisan. See, e.g., Braunwald et al., Unstable angina: diagnosis and management, Clinical practice guideline no. 10 (amended), AHCPR publication no. 94-0602. Rockville, Md.: Department of Health and Human Services, 1994; Yusuf et al., Lancet 352:507-514, 1998; Savonitto et al., JAMA 281:707-713, 1999; Klootwijk and Hamm, Lancet 353 (suppl II): 10-15, 1999.

In related aspects, one or more additional markers can be combined with a level of GPx-1 activity in a subject sample to increase the predictive value of GPx-1 as a prognostic indicator. The phrase “increases the predictive value” refers to the ability of two or more combined markers to improve the ability to predict a given outcome, in comparison to a prediction obtained from any of the prognostic indicators alone. For example, a level of GPx-1 activity of less than about 48.3 U/g hemoblobin may be correlated to an increased prognostic risk in the patient with a particular hazard ratio; and a cardiac troponin I level of Y ng/mL may independently be correlated to an increased prognostic risk in the patient with a second particular hazard ratio. But the presence of both a level of GPx-1 activity of less than about 48.3 U/g hemoblobin and a cardiac troponin I level of Y ng/mL in sample(s) obtained from the same patient may indicate a much higher (or lower) risk in the patient. Preferred additional prognostic indicators of the invention are one or more cardiac-specific troponins, myoglobin, TNF-α, IL-6, IL-18, fibrinogen, thrombus precursor protein (“TpP”), monocyte chemoattractant protein-1 (“MCP-1”), B-type natriuretic peptide (“BNP”), NT-proBNP, creatine kinase-MB, homocysteine, MMP-9, caspase-3, myeloperoxidase, sCD40L, and c-reactive protein, or markers related thereto. This list is not meant to be limiting, and additional markers for use as prognostic indicators are described hereinafter. In addition, non-polypeptide markers such as ST-segment depression or elevation, age, smoking status, diabetes, ejection fraction, hypertension, and/or prior MI may also be used as additional prognostic indicators that may be combined with a level of GPx-1 activity in a subject sample.

The skilled artisan will understand that the plurality of prognostic indicators need not be determined in the same sample, or even at the same time. For example, one prognostic indicator may not appear in serum samples until some time has passed from the onset of ACS. Nevertheless, combining, for example, a cardiac troponin I level taken at 1 hour with a level of GPx-1 activity obtained at 48 hours (or vice versa), may provide the skilled artisan with an increased predictive value in comparison to either measurement alone.

Additionally, the increased predictive value need not be an increased probability of an adverse outcome. For example, a level of GPx-1 activity in a subject sample taken at 1 hour may be indicative of a poor outcome, but when combined with a later level of GPx-1 activity, the temporal change in level of GPx-1 activity may indicate an improved (or worsened) prognosis in the subject.

In another aspect, the invention relates to methods for determining a prognostic panel comprising a plurality of prognostic markers, one of which is GPx-1, that can be used to assign a prognosis to a patient diagnosed with a cardiovascular condition, preferably an acute coronary syndrome. Once the plurality of markers has been determined, the levels of the various markers making up the panel can be measured in one or more patient sample(s), and then compared to the diagnostic levels determined for each marker, as described above.

It is another object of the invention to provide methods for diagnosing a subject as suffering from one or more cardiovascular conditions. In certain aspects, the present invention provides methods for identifying a level of GPx-1 activity that is associated with a particular cardiovascular condition, preferably an acute coronary syndrome. In related aspects, the level of GPx-1 activity in a sample obtained from a subject can be measured, and compared to a diagnostic level that is associated with a particular cardiovascular condition. By correlating the subject level to the prognostic level, a diagnosis can be assigned to the subject.

The phrase “determining the diagnosis” as used herein refers to methods by which the skilled artisan can determine the presence or absence of a particular disease or condition in a patient. The term “diagnosis” does not refer to the ability to determine the presence or absence of a particular disease or condition with 100% accuracy, or that a given course or outcome is more likely to occur than not. As discussed above, ROC curve analysis may be employed to assign a threshold value above which (or below which, depending on how a marker changes with the disease) the test is considered to be indicative of one state or condition (e.g., diseased) and below which the test is considered to be indicative of another state or condition (e.g., non-diseased). In certain embodiments, a level of GPx-1 activity is selected to exhibit at least about 70% sensitivity, more preferably at least about 80% sensitivity, even more preferably at least about 85% sensitivity, still more preferably at least about 90% sensitivity, and most preferably at least about 95% sensitivity, combined with at least about 70% specificity, more preferably at least about 80% specificity, even more preferably at least about 85% specificity, still more preferably at least about 90% specificity, and most preferably at least about 95% specificity. In particularly preferred embodiments, both the sensitivity and specificity are at least about 75%, more preferably at least about 80%, even more preferably at least about 85%, still more preferably at least about 90%, and most preferably at least about 95%. The term “about” in this context refers to +/−5% of a given measurement.

As discussed above with regard to prognostic indicators, the skilled artisan will understand that associating a diagnostic indicator with a predisposition to a particular outcome is a statistical analysis. Preferred confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001. The term “correlating,” as used herein in reference to the use of diagnostic indicators to assign a diagnosis, refers to comparing the presence or amount of the diagnostic indicator in a subject sample to its presence or amount in subjects known to suffer from, or known to be at risk of, a given condition; or in subjects known to be free of a given condition.

In particularly preferred embodiments, GPx-1 is applied to diagnose an acute coronary syndrome, selected from unstable angina, non-ST-elevation non-Q wave MI, ST-elevation non-Q wave MI, and transmural (Q-wave) MI. ACS can also be divided into non-ST-elevation ACS and ST-elevation ACS.

In still other aspects, one or more additional markers can be combined with a level of GPx-1 activity in a subject sample to diagnose one or more cardiovascular diseases. Preferred additional diagnostic indicators of the invention are one or more cardiac-specific troponins, myoglobin, TNF-α, IL-6, IL-18, fibrinogen, TpP, MCP-1, BNP, NT-proBNP, creatine kinase-MB, homocysteine, MMP-9, caspase-3, myeloperoxidase, sCD40L, and C-reactive protein, or markers related thereto. Again, this list is not meant to be limiting. In addition, non-polypeptide markers such as ST-segment depression, age, smoking status, diabetes, ejection fraction, hypertension, and/or prior MI may also be used as additional diagnostic indicators that may be combined with a level of GPx-1 activity in a subject sample.

It is yet another object of the invention to provide methods for determining and/or monitoring a treatment regimen for use in a patient diagnosed with a cardiovascular disease, most preferably an acute coronary syndrome. With regard to determining a treatment regimen, the methods preferably comprise determining a level of GPx-1 activity, which may be used alone or in combination with one or more additional markers as described herein, to determine a prognosis and/or diagnosis for a patient. One or more treatment regimens appropriate for the particular prognosis and/or diagnosis can then be used to treat the patient. With regard to monitoring a course of treatment, changes in a level of GPx-1 activity, which may be used alone or in combination with one or more additional markers as described herein, may be used to assess changes in the patient's health status resulting from a treatment regimen.

It is yet another object of the invention to provide kits for determining the prognosis and/or diagnosis of a patient diagnosed with a cardiovascular disease, most preferably an acute coronary syndrome. These kits preferably comprise devices and reagents for measuring a level of GPx-1 activity in a patient sample, and instructions for performing the assay. Optionally, the kits may contain one or more means, such as a threshold value, for converting a level of GPx-1 activity to a prognosis or diagnosis. Additionally, the kits may provide devices and reagents for determining one or more additional prognostic markers to be combined with a level of GPx-1 activity in a patient sample.

### BRIEF DESCRIPTION OF THE FIGURES

FIG. 1 shows the sequence of GPx-1 from Swiss-Prot accession number P07203. The signal sequence (residues 1-21) is shown in bold. The complete membrane bound form (residues 22-455) contains a putative extracellular domain (residues 22-211, underlined), a putative transmembrane domain (residues 212-234) and a putative cytoplasmic domain (residues 235-455, italicized).

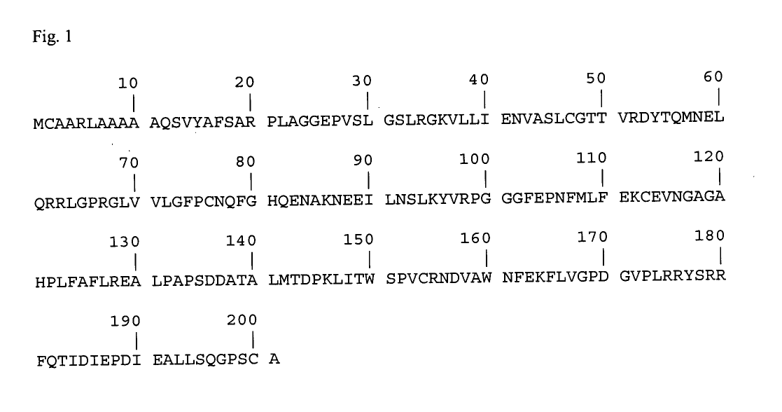

FIG. 2 shows Kaplan-Meier curves for cardiovascular events, according to GPx-1 quartile. The numbers of cardiovascular events were 33, 23, 16, and 11 in quartiles 1-4, respectively.

FIG. 3 shows age- and sex-adjusted hazard ratios for cardiovascular events according to the level of GPx-1 activity and smoking status. The interaction tested is between smoking category and GPx-1 activity (as a continuous variable). A high level of GPx-1 activity (hatched bars) was defined as more than the median value of 48.32 units per gram of hemoglobin, and a low level (solid bars) as 48.32 or fewer units per gram of hemoglobin. P for the interaction=0.007.

### DETAILED DESCRIPTION OF THE PREFERRED EMBODIMENTS

Patients presenting for medical treatment often exhibit one or a few primary observable changes in bodily characteristics or functions that are indicative of disease. Often, these “symptoms” are nonspecific, in that a number of potential diseases can present the same observable symptom or symptoms. A typical list of nonspecific symptoms in a cardiovasacular disease patient might include one or more of the following: shortness of breath (or dyspnea), chest pain, fever, dizziness, and headache. These symptoms can be common to a number of diseases, the number of which that must be considered by the clinician can be astoundingly broad.

Taking shortness of breath (referred to clinically as “dyspnea”) as an example, this symptom considered in isolation may be indicative of conditions as diverse as asthma, chronic obstructive pulmonary disease (“COPD”), tracheal stenosis, pulmonary injury, obstructive endobroncheal tumor, pulmonary fibrosis, pneumoconiosis, lymphangitic carcinomatosis, kyphoscoliosis, pleural effusion, amyotrophic lateral sclerosis, congestive heart failure, coronary artery disease, myocardial infarction, atrial fibrillation, cardiomyopathy, valvular dysfunction, left ventricle hypertrophy, pericarditis, arrhythmia, pulmonary embolism, metabolic acidosis, chronic bronchitis, pneumonia, anxiety, sepsis, aneurismic dissection, etc. See, e.g., Kelley's Textbook of Internal Medicine, 4^{th} Ed., Lippincott Williams & Wilkins, Philadelphia, Pa., 2000, pp. 2349-2354, “Approach to the Patient With Dyspnea”; Mulrow et al., J. Gen. Int. Med. 8: 383-92 (1993).

Similarly, chest pain, when considered in isolation, may be indicative of stable angina, unstable angina, myocardial ischemia, atrial fibrillation, myocardial infarction, musculoskeletal injury, cholecystitis, gastroesophageal reflux, pulmonary embolism, pericarditis, aortic dissection, pneumonia, anxiety, etc. Moreover, the classification of chest pain as stable or unstable angina (or even mild myocardial infarction) in cases other than definitive myocardial infarction is often completely subjective. The diagnosis, and in this case the distinction, is often made not by angiography, which may quantify the degree of arterial occlusion, but rather by a physician's interpretation of clinical symptoms.

Differential diagnosis refers to methods for diagnosing the particular disease(s) and/or condition(s) underlying the symptoms in a particular subject, based on a comparison of the characteristic features observable from the subject to the characteristic features of those potential diseases. Depending on the breadth of diseases and conditions that must be considered in the differential diagnosis, the types and number of tests that must be ordered by a clinician can be quite large. In the case of dyspnea for example, the clinician may order tests from a group that includes radiography, electrocardiography, exercise treadmill testing, blood chemistry analysis, echocardiography, bronchoprovocation testing, spirometry, pulse oximetry, esophageal pH monitoring, angiography, laryngoscopy, computed tomography, histology, cytology, magnetic resonance imaging, etc. See, e.g., Morgan and Hodge, Am. Fam. Physician 57: 711-16 (1998). The clinician must then integrate information obtained from a battery of tests, leading to a clinical diagnosis that most closely represents the range of symptoms and/or diagnostic test results obtained for the subject.

The present invention describes methods and compositions that can assist the clinician in performing differential diagnosis by assigning a diagnosis or a prognosis to a subject using one or more subject derived markers, one of which is GPx-1.

The term “marker” as used herein refers to proteins, polypeptides, phospholipids, small molecules, or other characteristics of one or more subjects to be used as targets for screening test samples obtained from subjects. “Proteins or polypeptides” used as markers in the present invention are contemplated to include any fragments thereof, in particular, immunologically detectable fragments. “Marker” as used herein may also include derived markers as defined below, and may also include such characteristics as patient's history, age, sex and race, for example.

The term “derived marker” as used herein refers to a value that is a function of one or more measured markers. For example, derived markers may be related to the change over a time interval in one or more measured marker values, may be related to a ratio of measured marker values, may be a marker value at a different measurement time, or may be a complex function such as a panel response function.

The term “related marker” as used herein refers to one or more fragments of a particular marker or its biosynthetic parent that may be detected as a surrogate for the marker itself or as independent markers. For example, human BNP is derived by proteolysis of a 108 amino acid precursor molecule, referred to hereinafter as BNP₁₋₁₀₈. Mature BNP, or “the BNP natriuretic peptide,” or “BNP-32” is a 32 amino acid molecule representing amino acids 77-108 of this precursor, which may be referred to as BNP₇₇₋₁₀₈. The remaining residues 1-76 are referred to hereinafter as BNP₁₋₇₆. Additionally, related markers may be the result of covalent modification of the parent marker, for example by further hydrolysis by proteases, oxidation of methionine residues, ubiquitination, cysteinylation, nitrosylation, glycosylation, etc.

Because production of marker fragments is an ongoing process that may be a function of, inter alia, the elapsed time between onset of an event triggering marker release into the tissues and the time the sample is obtained or analyzed; the elapsed time between sample acquisition and the time the sample is analyzed; the type of tissue sample at issue; the storage conditions; the quantity of proteolytic enzymes present; etc., it may be necessary to consider this degradation when both designing an assay for one or more markers, and when performing such an assay, in order to provide an accurate prognostic or diagnostic result. In addition, individual antibodies that distinguish amongst a plurality of marker fragments may be individually employed to separately detect the presence or amount of different fragments. The results of this individual detection may provide a more accurate prognostic or diagnostic result than detecting the plurality of fragments in a single assay. For example, different weighting factors may be applied to the various fragment measurements to provide a more accurate estimate of the amount of natriuretic peptide originally present in the sample.

Removal of polypeptide markers from the circulation often involves degradation pathways. Moreover, inhibitors of such degradation pathways may hold promise in treatment of certain diseases. See, e.g., Trindade and Rouleau, Heart Fail. Monit. 2: 2-7, 2001. However, the measurement of the polypeptide markers has focused generally upon measurement of the intact form without consideration of the degradation state of the molecules. Assays may be designed with an understanding of the degradation pathways of the polypeptide markers and the products formed during this degradation, in order to accurately measure the biologically active forms of a particular polypeptide marker in a sample. The unintended measurement of both the biologically active polypeptide marker(s) of interest and inactive fragments derived from the markers may result in an overestimation of the concentration of biologically active form(s) in a sample.

The failure to consider the degradation fragments that may be present in a clinical sample may have serious consequences for the accuracy of any diagnostic or prognostic method. Consider for example a simple case, where a sandwich immunoassay is provided for GPx-1, and a significant amount (e.g., 50%) of the GPx-1 originally present in the sample has now been degraded into smaller fragments. An immunoassay formulated with antibodies that bind a region common to the intact GPx-1 and the smaller fragment(s) may overestimate the amount of GPx-1 present in the sample by 2-fold, potentially resulting in a “false positive” result. Overestimation of certain form(s) present in a sample may also have serious consequences for patient management. Considering GPx-1 as an example again, the GPx-1 concentration may be used to determine if therapy is effective (e.g., by monitoring GPx-1 to see if an abnormal level is changing upon treatment, reflecting either an improving state, a static state, or a worsening state). The same “false positive” GPx-1 result discussed above may lead the physician to continue, increase, or modify treatment because of the false impression that current therapy is ineffective.

Likewise, it may be necessary to consider the complex state of one or more markers described herein. For example, troponin exists in muscle mainly as a “ternary complex” comprising three troponin polypeptides (T, I and C). But troponin I and troponin T circulate in the blood in forms other than the I/T/C ternery complex. Rather, each of (i) free cardiac-specific troponin I, (ii) binary complexes (e.g., troponin I/C complex), and (iii) ternary complexes all circulate in the blood. Furthermore, the “complex state” of troponin I and T may change over time in a patient, e.g., due to binding of free troponin polypeptides to other circulating troponin polypeptides. Immunoassays that fail to consider the “complex state” of a protein marker may not detect all of the marker present.

Preferably, the methods described hereinafter utilize one or more markers, including GPx-1, that are derived from the subject. The term “subject-derived marker” as used herein refers to protein, polypeptide, phospholipid, nucleic acid, prion, or small molecule markers that are expressed or produced by one or more cells of the subject. The presence, absence, amount, or change in amount of one or more markers may indicate that a particular disease is present, or may indicate that a particular disease is absent. Additional markers may be used that are derived not from the subject, such as molecules expressed by pathogenic or infectious organisms that are correlated with a particular disease, race, time since onset, sex, etc. Such markers are preferably protein, polypeptide, phospholipid, nucleic acid, prion, or small molecule markers that identify the infectious diseases described above. Exemplary subject derived markers are described herein, and in PCT application no. US03/41453, filed on Dec. 23, 2003 (WO 04059293), of which is hereby incorporated by reference in its entirety.

The term “marker related to myocardial injury” refers to subject-derived markers that are known in the art to be derived from cardiac tissue and that are elevated in the circulation of subjects suffering from damage to the myocardium. Preferred markers of cardiac injury for use in the methods described herein comprise, for example, annexin V, β-enolase, cardiac troponin I (total, free of other troponin polypeptides, and/or complexed with other troponin polypeptides), cardiac troponin T (total, free of other troponin polypeptides, and/or complexed with other troponin polypeptides), creatine kinase-MB, glycogen phosphorylase-BB, heart-type fatty acid binding protein, phosphoglyceric acid mutase-MB, S-100ao, myoglobin, actin, myosin, and lactate dehydrogenase, or markers related thereto. This list is not meant to be limiting.

The term “marker related to apoptosis” refers to subject-derived markers that are elevated in the circulation due to apoptotic processes. Preferred marker(s) related to apoptosis for use in the methods described herein comprise, for example, one or more marker(s) selected from the group consisting of spectrin, cathepsin D, caspase 3, cytochrome c, s-acetyl glutathione, and ubiquitin fusion degradation protein 1 homolog, or markers related thereto. This list is not meant to be limiting.

The term “marker related to blood pressure regulation” refers to subject-derived markers that are known in the art to affect blood pressure regulation. Preferred marker(s) related to blood pressure regulation for use in the methods described herein comprise, for example, one or more marker(s) selected from the group consisting of atrial natriuretic peptide (“ANP”), pro-ANP, B-type natriuretic peptide (“BNP”), NT-pro BNP, pro-BNP C-type natriuretic peptide (“CNP”), pro-CNP, urotensin II, arginine vasopressin, aldosterone, angiotensin I, angiotensin II, angiotensin III, bradykinin, calcitonin, procalcitonin, calcitonin gene related peptide, adrenomedullin, calcyphosine, endothelin-2, endothelin-3, renin, and urodilatin, or markers related thereto. This list is not meant to be limiting.

The term “marker related to inflammation” refers to subject-derived markers that are known in the art to mediate or promote inflammation, activate the complement cascade, and/or stimulate chemotaxis of phagocytes. Preferred marker(s) markers related to inflammation for use in the methods described herein comprise, for example, one or more marker(s) selected from the group consisting of hepcidin, HSP-60, HSP-65, HSP-70, asymmetric dimethylarginine (an endogenous inhibitor of nitric oxide synthase), matrix metalloproteins 11, 3, and 9, defensin HBD 1, defensin HBD 2, serum amyloid A, oxidized LDL, insulin like growth factor, transforming growth factor β, e-selectin, glutathione-S-transferase, hypoxia-inducible factor-1α, inducible nitric oxide synthase (“I-NOS”), intracellular adhesion molecule, lactate dehydrogenase, monocyte chemoattractant peptide-1 (“MCP-1”), n-acetyl aspartate, prostaglandin E2, receptor activator of nuclear factor (“RANK”) ligand, lipopolysaccharide binding protein (“LBP”), high mobility group protein-1 (“HMG-1” or “HMGB1”), cystatin C, cell adhesion molecules such as vascular cell adhesion molecule (“VCAM”), intercellular adhesion molecule-1 (“ICAM-1”), intercellular adhesion molecule-2 (“ICAM-2”), and intercellular adhesion molecule-3 (“ICAM-3”), myeloperoxidase (“MPO”), C-reactive protein (“CRP”), interleukins such as IL-1β, IL-6, and IL-8, interleukin-1 receptor agonist, monocyte chemoattractant protein-1, lipocalin-type prostaglandin D synthase, mast cell tryptase, eosinophil cationic protein, haptoglobin, tumor necrosis factor α (“TNF-α”), tumor necrosis factor β, Fas ligand, soluble Fas (Apo-1), TRAIL, TWEAK, fibronectin, macrophage migration inhibitory factor (MIF), and vascular endothelial growth factor (“VEGF”), or markers related thereto. The term “acute phase reactants” as used herein refers to proteins whose concentrations are elevated in response to stressful or inflammatory states that occur during various insults that include infection, injury, surgery, trauma, tissue necrosis, and the like. Acute phase reactant expression and serum concentration elevations are not specific for the type of insult, but rather as a part of the homeostatic response to the insult. This list is not meant to be limiting.

The term “marker related to coagulation and hemostasis” refers to subject-derived markers that are known in the art to be associated with clot presence, or any condition that causes or is a result of fibrinolysis activation. Preferred marker(s) related to coagulation and hemostasis for use in the methods described herein comprise, for example, one or more marker(s) selected from the group consisting of plasmin, fibrinogen, thrombus precursor protein, D-dimer, β-thromboglobulin, platelet factor 4, fibrinopeptide A, platelet-derived growth factor, prothrombin fragment 1+2, plasmin-α2-antiplasmin complex, thrombin-antithrombin III complex, P-selectin, thrombin, and von Willebrand factor, tissue factor, or markers related thereto. This list is not meant to be limiting.

The term “test sample” as used herein refers to a sample of bodily fluid obtained for the purpose of diagnosis, prognosis, or evaluation of a subject of interest, such as a patient. In certain embodiments, such a sample may be obtained for the purpose of determining the outcome of an ongoing condition or the effect of a treatment regimen on a condition. Preferred test samples include blood, serum, plasma, cerebrospinal fluid, urine, saliva, sputum, and pleural effusions. In addition, one of skill in the art would realize that some test samples would be more readily analyzed following a fractionation or purification procedure, for example, separation of whole blood into serum or plasma components.

As used herein, a “plurality” refers to at least two. Preferably, a plurality refers to at least 3, more preferably at least 5, even more preferably at least 10, even more preferably at least 15, and most preferably at least 20. In particularly preferred embodiments, a plurality is a large number, i.e., at least 100.

The term “subject” as used herein refers to a human or non-human animal. Thus, the methods and compositions described herein are applicable to both human and veterinary disease. Further, while a subject is preferably a living animal, the invention described herein may be used in post-mortem analysis as well. Preferred subjects are “patients,” i.e., living humans that are receiving or being evaluated for medical care. This includes persons with no defined illness who are being investigated for signs of pathology.

The phrase “clinical outcome” as used herein refers to the future course of a disease suffered by a subject. Such a clinical outcome may be adverse (e.g., future morbidity or mortality) or may be beneficial (e.g., future improvement in health). In the case of a cardiovascular disease such as ACS for example, an adverse outcome could be a future MI (fatal and/or non-fatal), future stroke (fatal and/or non-fatal), future congestive heart failure, future stable angina, future unstable angina, future need for rehospitalization (that is, the need to readmit a patient for hospital-based treatment following clinical improvement in the patient's present condition sufficient to warrant release from an in-patient setting), future need for coronary revascularization (that is, surgical intervention to improve blood flow to the heart, e.g., by coronary artery bypass grafting, insertion of a stent, percutaneous coronary intervention, etc.), or future death. A clinical outcome is preferably measured within 5 years of the measurement of a level of GPx-1 activity used to assign a prognosis. A clinical outcome is said to occur within the “near term” if it occurs within about 2 years, preferably within about 12 months, more preferably about 9 months, still more preferably about 6 months, even more preferably about 3 months, and most preferably within about 1 month of the measurement of a level of GPx-1 activity used to assign a prognosis.

The term “discrete” as used herein refers to areas of a surface that are non-contiguous. That is, two areas are discrete from one another if a border that is not part of either area completely separates each of the two areas.

The term “independently addressable” as used herein refers to discrete areas of a surface from which an independent signal may be obtained.

The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3^{rd} Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain, and (vi) an isolated complementarity determining region (CDR). Single chain antibodies are also included by reference in the term “antibody.”

Use of GPx-1 as a Prognostic Marker

As described herein, levels of GPx-1 activity may be strongly predictive of the future clinical course of patients with one or more cardiovascular conditions. In patients with coronary artery disease, a low level of activity of erythrocyte intracellular glutathione peroxidase 1 is independently associated with an increased risk of cardiovascular events. Furthermore, the combination of an erythrocyte level of GPx-1 activity with other markers (e.g., markers related to myocardial injury, markers related to inflammation, markers related to blood pressure regulation, markers related to apoptosis, markers related to coagulation, etc.) may improve the predictive value of GPx-1. Likewise, certain characteristics such as ST-segment depression, age, smoking status, lipid levels, diabetes, ejection fraction, hypertension, and/or prior MI may also be used as additional prognostic indicators that may be combined with a level of GPx-1 activity in a subject sample.

Oxidative stress may be defined as an imbalance between the production and degradation of reactive oxygen species such as superoxide anion, hydrogen peroxide, lipid peroxides, and peroxynitrite. Enzymatic inactivation of reactive oxygen species is achieved mainly by glutathione peroxidase, superoxide dismutase, and catalase, atthough glutathione and the glutathione peroxidases constitute the principal antioxidant defense system in mammalian cells. There are at least four different glutathione peroxidases, all of which contain selenocysteine at their active sites. Glutathione peroxidase 1 is a ubiquitous intracellular form within most cells, including erythrocytes. In mice, glutathione peroxidase 1 deficiency reportedly results in abnormal vascular and cardiac function and structure.

Erythrocyte GPx-1 may be obtained from blood (drawn under standardized conditions in the presence of anticoagulants such as heparin, citrate, or EDTA) following hemolysis of the erythrocytes to release the intracellular enzyme. Standard methods for erythrocyte hemolysis comprise collection of erythrocytes by centrifugation, followed by exposure to hypotonic conditions (e.g., resuspension in 4 volumes of water at ˜4° C., followed by separation of cellular debris from the lysate by centrifugation. Other methods for obtaining erythrocyte GPx-1 will be apparent to those of skill in the art. Erythrocyte GPx-1 may then be assayed by functional enzymatic assays that are well known in the art. See, e.g., Paglia and Valentine, J. Lab. Clin. Med. 70: 158-69, 1967, which is hereby incorporated by reference in its entirety. Commercially available assays (e.g., those available from Cayman Chemical and Randox Laboratories) may be employed for this purpose. In the alternative, erythrocyte GPx-1 may be assayed using standard immunoassay methods as described hereinafter.

In the exemplary embodiments described hereinafter, erythrocyte GPx-1 activity was measured in a prospective cohort of patients with angiographically documented coronary artery disease, which were then followed for future fatal and nonfatal cardiovascular events. Erythrocyte GPx-1 (measured by enzymatic activity) was inversely associated with both future fatal and nonfatal events. The risk of a cardiovascular event among subjects in the highest quartile of erythrocyte GPx-1 activity was approximately 30 percent of that among subjects in the lowest quartile. This difference did not change appreciably after adjustment for most potential confounders, indicating that the relation between GPx-1 and future cardiovascular events is independent of other risk factors and clinical features. Erythrocyte GPx-1 may be considered a surrogate marker for cellular glutathione peroxidase 1 activity in general. Thus, the present invention is not limited to measurement of the erythrocyte enzyme, and any cellular population collected from a subject may be assayed for intracellular GPx-1.

The variability in erythrocyte GPx-1 activity within an individual patient has been reported to be about half the variability between patients (see, e.g., Andersen et al., Clin. Chem. 43: 562-68, 1997). The causes of variability between patients are not well established. The level of GPx-1 activity is also higher in women than in men, as has been described previously. See, e.g., Massafra et al., Clin. Endocrinol. 57: 663-67, 2002.

Smoking consistently reduces GPx-1 activity, whereas the effect of commonly used drugs appears to be negligible. Cigarette smoking is strongly associated with dysfunctional vasomotor responses, diminished nitric oxide levels, and time-dependent decreases in the content of endothelial nitric oxide synthase messenger RNA. In accordance with previous studies, glutathione peroxidase 1 activity was decreased in smokers and former smokers. However, the association between low levels of glutathione peroxidase 1 activity and high cardiovascular risk was also observed in smokers. Therefore, measurement of glutathione peroxidase 1 may be used to identify smokers who are at highest risk for cardiovascular events.

In various embodiments, GPx-1 is applied to determine the prognosis in a subject suffering from cardiovascular conditions as defined herein. In preferred embodiments, GPx-1 is applied to determine a prognosis in a subject suffering from an acute coronary syndrome. In various embodiments, the methods are applied to determine a prognosis in a subject suffering from non-ST-elevation ACS, ST-elevation ACS, unstable angina, non-ST-elevation non-Q wave MI, ST-elevation non-Q wave MI, and/or transmural (Q-wave) MI. In still other embodiments, GPx-1 is applied to determine a prognosis in a clinically normal subject.

While described in exemplary embodiments in terms of cardiovascular conditions, GPx-1 may also be applied to prognosis according to the methods described herein in other diseases and conditions in which oxidative stress is manifested. Such diseases and conditions include stroke, diabetes, cancer, arthritis, Alzheimer's dementia, macular degeneration, lupus, multiple sclerosis, fibromyalgia, AIDS, ulcerative colitis, Crohn's disease, hepatitis B and C, psoriasis, chronic fatigue, etc. This list is not meant to be limiting.

Use of GPx-1 as a Diagnostic Marker

As in the case of prognosis, GPx-1 may also be applied to the diagnosis of cardiovascular conditions as defined herein. In preferred embodiments, GPx-1 is preferably applied to diagnosis of an acute coronary syndrome. In various embodiments, the methods are applied to diagnose a subject suffering from non-ST-elevation ACS, ST-elevation ACS, unstable angina, non-ST-elevation non-Q wave MI, ST-elevation non-Q wave MI, and/or transmural (Q-wave) MI. In addition, one or more GPx-1 may also be applied to diagnosis according to the methods described herein in other diseases and conditions in which oxidative stress is manifested as described herein.

Combination of GPx-1 with Other Markers

In traditional methods to evaluate marker levels in the diagnosis or prognosis of disease, a “threshold” for a marker of interest is typically established, and the concentration of that marker in a sample is compared to that threshold amount; an amount greater than the pre-established threshold is indicative of one state (e.g., disease), and an amount less than the pre-established threshold is indicative of another state (e.g., normal). For example, the American Heart Association has stated that a cardiac troponin I concentration greater that the 99th percentile concentration in the normal population should be used to rule in myocardial infarction.

In certain preferred embodiments, a diagnosis and/or prognosis may be assigned based on the contributions of a plurality of markers. When combining markers, a threshold may be established for each marker of interest. The concentration of each marker in a sample is then compared to its appropriate threshold amount. A particular diagnosis/prognosis may be assigned, depending on the outcome of each comparison. One skilled in the art will recognize that univariate analysis of markers can be performed and the data from the univariate analyses of multiple markers can be combined to form panels of markers to differentiate different disease conditions. In addition, multivariate methods for combining markers are well known to those of skill in the art. See, e.g., Di Fabio et al., Dig. Surg. 21:128-133, 2004; Latini et al., Eur. Heart J. 25(4):292-9, 2004.

In certain embodiments, the present invention may utilize an evaluation of the plurality of markers as a unitary whole. In a simple example, the ratio of two or more markers, rather than an absolute amount of the markers, may be used to determine a diagnosis/prognosis. Even more preferably, however, a particular “fingerprint” pattern of changes in such a panel of markers may, in effect, act as a specific diagnostic or prognostic indicator. Methods for determining a “panel response value” that integrates a plurality of marker concentrations into a single result are described in International Application No. US03/41426, filed Dec. 23, 2003.

The diagnostic and/or prognostic panels of the present invention may comprise 2, 3, 4, 5, 6, 7, 8, 9, 10, 15, 20, or more or individual markers. Preferred panels comprise, in addition to GPx-1, one or more additional markers independently selected from the group consisting of specific markers of cardiac injury, markers related to blood pressure regulation, markers related to inflammation, markers related to coagulation and hemostasis, and markers related to apoptosis. Exemplary markers in each of these groups are described herein. Particularly preferred panels comprise one or more of the following: caspase-3, thrombus precursor protein, creatine kinase-MB, total cardiac troponin I and/or T, free cardiac troponin I and/or T, complexed cardiac troponin I and/or T, myoglobin, B-type natriuretic peptide, NT-proBNP, C-reactive protein, D-dimer, and myeloperoxidase, or markers related thereto.

These markers may be combined in various combinations. For example, preferred panels may comprise 2, 3, 4, 5, or more of the following markers: GPx-1, B-type natriuretic peptide NT-proBNP, creatine kinase-MB, total cardiac troponin I, total cardiac troponin T, C-reactive protein, D-dimer, and myoglobin, or markers related thereto.

Assay Measurement Strategies

Numerous methods and devices are well known to the skilled artisan for the detection and analysis of the markers of the instant invention. With regard to polypeptides or proteins in patient test samples, immunoassay devices and methods are often used. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety, including all tables, figures and claims. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety, including all tables, figures and claims. One skilled in the art also recognizes that robotic instrumentation including but not limited to Beckman Access, Abbott AxSym, Roche ElecSys, Dade Behring Stratus systems are among the immunoassay analyzers that are capable of performing the immunoassays taught herein.

In the exemplary embodiments described hereinafter, GPx-1 is measured in terms of its enzymatic activity. In other emobodiments, one or more markers are analyzed using an immunoassay, although other methods are well known to those skilled in the art (for example, measurement by capillary electrophoresis; measurement by chromatographic methods; measurement by mass spectrometry; measurement of marker RNA levels). The presence or amount of a marker is generally determined using antibodies specific for each marker and detecting specific binding. Any suitable immunoassay may be utilized, for example, enzyme-linked immunoassays (ELISA), radioimmunoassays (RIAs), competitive binding assays, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies specific for the markers is also contemplated by the present invention. The antibodies could be immobilized onto a variety of solid supports, such as magnetic or chromatographic matrix particles, the surface of an assay place (such as microtiter wells), pieces of a solid substrate material or membrane (such as plastic, nylon, paper), and the like. An assay strip could be prepared by coating the antibody or a plurality of antibodies in an array on solid support. This strip could then be dipped into the test sample and then processed quickly through washes and detection steps to generate a measurable signal, such as a colored spot.

The analysis of a plurality of markers may be carried out separately or simultaneously with one test sample. For separate or sequential assay of markers, suitable apparatuses include clinical laboratory analyzers such as the ElecSys (Roche), the AxSym (Abbott), the Access (Beckman), the ADVIA® CENTAUR® (Bayer) immunoassay systems, the NICHOLS ADVANTAGE® (Nichols Institute) immunoassay system, etc. Preferred apparatuses or protein chips perform simultaneous assays of a plurality of markers on a single surface. Particularly useful physical formats comprise surfaces having a plurality of discrete, adressable locations for the detection of a plurality of different analytes. Such formats include protein microarrays, or “protein chips” (see, e.g., Ng and Ilag, J. Cell Mol. Med. 6: 329-340 (2002)) and certain capillary devices (see, e.g., U.S. Pat. No. 6,019,944). In these embodiments, each discrete surface location may comprise antibodies to immobilize one or more analyte(s) (e.g., a marker) for detection at each location. Surfaces may alternatively comprise one or more discrete particles (e.g., microparticles or nanoparticles) immobilized at discrete locations of a surface, where the microparticles comprise antibodies to immobilize one analyte (e.g., a marker) for detection.

Several markers may be combined into one test for efficient processing of a multiple of samples. In addition, one skilled in the art would recognize the value of testing multiple samples (for example, at successive time points) from the same individual. Such testing of serial samples will allow the identification of changes in marker levels over time. Increases or decreases in marker levels, as well as the absence of change in marker levels, would provide useful information about the disease status that includes, but is not limited to identifying the approximate time from onset of the event, the presence and amount of salvagable tissue, the appropriateness of drug therapies, the effectiveness of various therapies as indicated by reperfusion or resolution of symptoms, differentiation of the various types of ACS, identification of the severity of the event, identification of the disease severity, and identification of the patient's outcome, including risk of future events.

A panel consisting of the markers referenced above may be constructed to provide relevant information related to differential diagnosis and/or prognosis. Such a panel may be constucted using 1, 2, 3, 4, 5, 6, 7, 8, 9, 10, 15, 20, or more or individual markers. The analysis of a single marker or subsets of markers comprising a larger panel of markers could be carried out by one skilled in the art to optimize clinical sensitivity or specificity in various clinical settings. These include, but are not limited to ambulatory, urgent care, critical care, intensive care, monitoring unit, inpatient, outpatient, physician office, medical clinic, and health screening settings. Furthermore, one skilled in the art can use a single marker or a subset of markers comprising a larger panel of markers in combination with an adjustment of the diagnostic threshold in each of the aforementioned settings to optimize clinical sensitivity and specificity. The clinical sensitivity of an assay is defined as the percentage of those with the disease that the assay correctly predicts, and the specificity of an assay is defined as the percentage of those without the disease that the assay correctly predicts (Tietz Textbook of Clinical Chemistry, 2^{nd} edition, Carl Burtis and Edward Ashwood eds., W.B. Saunders and Company, p. 496).

The analysis of markers could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings.

In another embodiment, the present invention provides a kit for the analysis of markers. Such a kit preferably comprises devises and reagents for the analysis of at least one test sample and instructions for performing the assay. Optionally the kits may contain one or more means for using information obtained from immunoassays performed for a marker panel to rule in or out certain diagnoses.

Measures of test accuracy may be obtained as described in Fischer et al., Intensive Care Med. 29: 1043-51, 2003; Zhou et al., Statistical Methods in Diagnostic Medicine, John Wiley & Sons, 2002; and Motulsky, Intuitive Biostatistics, Oxford University Press, 1995; and other publications well known to those of skill in the art, and used to determine the effectiveness of a given marker or panel of markers. These measures include sensitivity and specificity, predictive values, likelihood ratios, diagnostic odds ratios, hazard ratios, and ROC curve areas. As discussed above, suitable tests may exhibit one or more of the following results on these various measures:

A ROC curve area of greater than about 0.5, more preferably greater than about 0.7, still more preferably greater than about 0.8, even more preferably greater than about 0.85, and most preferably greater than about 0.9; a positive or negative likelihood ratio of at least about 1.1 or more or about 0.91 or less, more preferably at least about 1.25 or more or about 0.8 or less, still more preferably at least about 1.5 or more or about 0.67 or less, even more preferably at least about 2 or more or about 0.5 or less, and most preferably at least about 2.5 or more or about 0.4 or less; an odds ratio of at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less; and/or a hazard ratio of at least about 1.1 or more or about 0.91 or less, more preferably at least about 1.25 or more or about 0.8 or less, still more preferably at least about 1.5 or more or about 0.67 or less, even more preferably at least about 2 or more or about 0.5 or less, and most preferably at least about 2.5 or more or about 0.4 or less.

Measures of diagnostic accuracy such as those discussed above are often reported together with confidence intervals or p values. These may be calculated by methods well known in the art. See, e.g., Dowdy and Wearden, Statistics for Research, John Wiley & Sons, New York, 1983. Preferred confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001.

Selection of Antibodies

The generation and selection of antibodies may be accomplished several ways. For example, one way is to purify polypeptides of interest or to synthesize the polypeptides of interest using, e.g., solid phase peptide synthesis methods well known in the art. See, e.g., Guide to Protein Purification, Murray P. Deutcher, ed., Meth. Enzymol. Vol 182 (1990); Solid Phase Peptide Synthesis, Greg B. Fields ed., Meth. Enzymol. Vol 289 (1997); Kiso et al., Chem. Pharm. Bull. (Tokyo) 38: 1192-99, 1990; Mostafavi et al., Biomed. Pept. Proteins Nucleic Acids 1: 255-60, 1995; Fujiwara et al., Chem. Pharm. Bull. (Tokyo) 44: 1326-31, 1996. The selected polypeptides may then be injected, for example, into mice or rabbits, to generate polyclonal or monoclonal antibodies. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)).

In addition, numerous publications have reported the use of phage display technology to produce and screen libraries of polypeptides for binding to a selected target. See, e.g, Cwirla et al., Proc. Natl. Acad. Sci. USA 87, 6378-82, 1990; Devlin et al., Science 249, 404-6, 1990, Scott and Smith, Science 249, 386-88, 1990; and Ladner et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means. See, e.g., U.S. Pat. No. 6,057,098, which is hereby incorporated in its entirety, including all tables, figures, and claims.

The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies is then placed into the respective microtiter wells and incubated for about 30 min to 2 h. The microtiter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized polypeptide(s) are present.

The antibodies so identified may then be further analyzed for affinity and specificity in the assay design selected. In the development of immunoassays for a target protein, the purified target protein acts as a standard with which to judge the sensitivity and specificity of the immunoassay using the antibodies that have been selected. Because the binding affinity of various antibodies may differ; certain antibody pairs (e.g., in sandwich assays) may interfere with one another sterically, etc., assay performance of an antibody may be a more important measure than absolute affinity and specificity of an antibody.

Those skilled in the art will recognize that many approaches can be taken in producing antibodies or binding fragments and screening and selecting for affinity and specificity for the various polypeptides, but these approaches do not change the scope of the invention.

Selecting and/or Monitoring a Treatment Regimen

The appropriate treatments for various types of cardiovascular disease may be large and diverse. However, once a diagnosis is obtained, the clinician can readily select a treatment regimen that is compatible with the diagnosis. Accordingly, the present invention provides methods of early differential diagnosis to allow for appropriate intervention in acute time windows. The skilled artisan is aware of appropriate treatments for numerous diseases discussed in relation to the methods of diagnosis described herein. See, e.g., Merck Manual of Diagnosis and Therapy, 17^{th} Ed. Merck Research Laboratories, Whitehouse Station, N.J., 1999.

Upon treatment, changes in the predicted prognosis of a patient may be monitored by the methods described herein. Any improvement (or lack thereof) may be assessed, and further clinical decisions made based (at least partly) upon this information. The skilled artisan will understand that additional information on health status from other clinical tests (e.g., electrocardiography, exercise treadmill testing, blood chemistry analysis, echocardiography, bronchoprovocation testing, spirometry, pulse oximetry, esophageal pH monitoring, angiography, laryngoscopy, computed tomography, histology, cytology, magnetic resonance imaging) may also be used to supplement the monitoring features of the present invention.

### EXAMPLES

The following examples serve to illustrate the present invention. These examples are in no way intended to limit the scope of the invention.

### Example 1

### Study Population

A detailed description of the design of the AtheroGene study has been outlined previously. See, e.g., Blankenberg et al., Circulation 106:24-30, 2002. Briefly, Between November 1996 and December 1997, 732 patients referred to the Department of Medicine II of the Johannes Gutenberg University in Mainz, Germany, with suspected coronary artery disease were enrolled in the AtheroGene registry. Fourteen patients with acute myocardial infarction and 75 patients in whom GPx-1 activity could not be determined immediately were excluded from the present analysis. Thus, the final study cohort consisted of 643 patients, 133 with symptoms of unstable angina and 510 with symptoms of stable angina. Coronary angiography was performed in all patients. Relevant coronary artery disease, defined by greater than 30 percent stenosis in at least one major coronary artery, was detected in 558 patients. The exclusion criteria were evidence of hemodynamically significant valvular heart disease, surgery or trauma within the previous month, known cardiomyopathy, known cancer, febrile conditions, or use of oral anticoagulant therapy within the previous four weeks.

Patients who were receiving dietary treatment or medication for diabetes or whose current fasting blood glucose level was above 125 mg per deciliter were identified as having diabetes mellitus. Patients who had received antihypertensive treatment or who had received a diagnosis of hypertension (blood pressure above 160/90 mm Hg) were identified as having hypertension. Patients were classified as currently smoking, as having smoked in the past (if they had stopped more than 4 weeks and less than 40 years earlier), or as never having smoked (if they had never smoked or had stopped 40 or more years earlier).

Among the 643 patients, 636 (98.9 percent) were followed for a median of 4.7 years (maximum, 5.4). There were 64 deaths from cardiovascular causes, 21 deaths from other causes, and 19 nonfatal myocardial infarctions. Information about the causes of death and clinical events was obtained from hospital and general practitioner charts.

The study was approved by the ethics committee of the University of Mainz. Participation was voluntary, and each patient gave written informed consent.

### Example 2

### Laboratory Methods

Blood was drawn under standardized conditions before coronary angiography was performed. GPx-1 activity and superoxide dismutase activity were determined in washed red cells obtained immediately after sampling from whole blood anticoagulated with EDTA. Hemolyzed cells were stored frozen for up to one week; freezing did not lead to changes in enzyme activity. GPx-1 activity was measured using the commercially available Ransel test kit (Randox). The intraassay and interassay coefficients of variation were 6.7 percent and 9.9 percent, respectively.

Superoxide dismutase activity was determined by the following method. Superoxide radicals generated by the xanthine oxidase reaction convert 1-(4-iodophenyl)-3-(4-nitrophenol)-5-phenyltetrazolium chloride quantitatively to a formazan dye (Ransod test kit, Randox). Conversion of superoxide radicals to hydrogen peroxide by superoxide dismutase inhibits dye formation and serves as a measure of superoxide dismutase activity. The intraassay and interassay coefficients of variation were 5.1 percent and 5.5 percent, respectively.

Serum lipid levels (the levels of total cholesterol, triglycerides, and high-density lipoprotein cholesterol) were measured immediately by routine methods; low-density lipoprotein cholesterol was calculated by the Friedewald formula.

For all other biologic markers measured in the study, plasma and serum were stored at −80° C. until analysis, which was performed after a mean of 1.5 years of storage time. C-reactive protein was measured with a highly sensitive, latex-particle-enhanced immunoassay (Roche Diagnostics; range of detection, 0.1 to 20 mg per liter; interassay coefficient of variation, 1.0 percent for values of 15 mg per liter and 6.5 percent for values below 4 mg per liter). Interleukin-6 and soluble ICAM-1 were measured with commercially available immunoassays (EASIA, Biosource Europe). Homocysteine was measured by high-pressure liquid chromatography (interassay coefficient of variation, 7.1 percent) and selenium by carbon-furnace atomic-absorption spectrometry with Zeeman compensation, as previously described (Oster and Prellwitz, Clin. Chem. Acta 124: 277-91, 1982).

### Example 3

### Statistical Analysis

The mean levels and proportions of base-line cardiovascular risk factors were calculated for study participants in whom a cardiovascular event subsequently occurred and in those without such an event. The significance of differences between the means for the two groups was assessed with Student's t-test, and the significance of differences in proportions was tested with the chi-square statistic. Variables with a skewed distribution were presented as medians, and the Wilcoxon rank-sum test was applied. The cumulative event plots according to quartile of GPx-1 activity were estimated by the Kaplan-Meier method and compared with use of the log-rank test. In all survival analyses, the end point was death from cardiovascular causes or nonfatal myocardial infarction. Data from patients who died from other causes were censored at the time of death. Hazard ratios for future coronary events according to quartile of GPx-1 activity were estimated by Cox regression models adjusted for potential confounders. Three adjusted models were constructed. We adjusted first for age and sex and second for other traditional risk factors. The final model included clinical and therapeutic variables as well as C-reactive protein, homocysteine, and creatinine.

To evaluate the combined effect of GPx-1 activity and smoking status on cardiovascular risk, we divided the study participants into six groups according to whether the GPx-1 activity was above the median or at or below the median and according to their smoking-status category. In these analyses, Cox regression was used to assess simultaneously the risk of future cardiovascular events in each of the six groups, with the group of patients whose GPx-1 activity was above the median and who had never smoked as the reference group. The hypothesis that smoking and the level of GPx-1 activity had an interactive effect on the risk of future cardiovascular events was formally tested in a Cox regression model that included a term for the multiplicative interaction of smoking (across the three categories of smoking status) and GPx-1 activity. The hazard ratios and their 95 percent confidence intervals are reported. The P values are two-sided; a P value of less than 0.05 was considered to indicate statistical significance. All computations were carried out with SPSS software, version 10.07.

### Example 4

### Evaluation of GPx-1 and Cardiovascular Risk Factors

Table 1 gives the base-line characteristics of the 83 study participants who subsequently died from cardiovascular causes or had a nonfatal myocardial infarction and the 553 who did not have a cardiovascular event, including the results of base-line blood-chemistry analysis. GPx-1 activity was normally distributed among the study participants. It ranged from 7.4 to 99.6 units per gram of hemoglobin, with a mean (±SD) of 49.2±11.6, a median of 48.3, and an interquartile range of 42.0 to 56.3 units per gram of hemoglobin. The base-line level of GPx-1 activity was significantly lower among those who died from cardiac causes or had a nonfatal myocardial infarction than among those who did not. This result was stable when data from the subgroups with fatal events (64 patients) and nonfatal events (19 patients) were analyzed separately (45.7±13.5 and 44.1±10.4 units per gram of hemoglobin, respectively, vs. 49.8±11.3 units per gram of hemoglobin for those who did not have cardiovascular events or die from noncardiovascular causes; P=0.009 and P=0.03, respectively). The base-line level of GPx-1 activity in patients who died from noncardiovascular causes (21 patients) was the same as that in event-free patients (49.7±11.1 vs. 49.8±11.3 units per gram of hemoglobin).

TABLE 1

| | Patients without a | Patients with a | |
|---|---|---|---|
| | Cardiovascular | Cardiovascular | |
| Characteristic | Event (N = 553) | Event (N = 83) | P Value |
| Age (yr) | 60.9 ± 10.1 | 67.0 ± 7.8 | <0.001 |
| Male sex (%) | 72.0 | 73.5 | 0.77 |
| Body-mass index | 26.8 ± 3.4 | 26.4 ± 4.1 | 0.45 |
| Diabetes (%) | 23.5 | 42.2 | <0.001 |
| Hypertension | 68.0 | 68.7 | 0.90 |
| Smoking status (%) | | | 0.07 |
| Never smoked | 42.0 | 31.3 | |
| Formerly smoked | 45.2 | 51.8 | |
| Currently smoke | 12.8 | 16.9 | |
| Disease in 2 or more | 63.5 | 80.7 | 0.002 |
| vessels | | | |
| History of myocardial | 46.1 | 45.8 | 0.95 |
| infarction | | | |
| Revascularization (%) | 53.3 | 59.0 | 0.33 |
| Left ventricular ejection | 63.4 ± 14.0 | 53.0 ± 20.2 | <0.001 |
| fraction | | | |
| Beta-blocker medication | 51.9 | 43.4 | 0.15 |
| (%) | | | |
| Statin medication (%) | 24.1 | 10.8 | 0.007 |
| Antioxidant enzymes and | | | |
| cofactors | | | |
| GPx-1 (U/g of | 49.8 ± 11.3 | 45.3 ± 12.9 | <0.001 |
| hemoglobin) | | | |
| Superoxide dismutase | | | 0.99 |
| (U/g of hemoglobin) | | | |
| Median | 9.9 | 9.7 | |
| Interquartile range | 8.7-11.4 | 8.5-11.6 | |
| Selenium (ng/mL) | 74.5 ± 33.5 | 69.5 ± 32.6 | 0.26 |
| Lipid variables | | | |
| LDL cholesterol | 140.6 ± 38.7 | 144.6 ± 40.7 | 0.45 |
| (mg/dL) | | | |
| HDL cholesterol | 47.1 ± 15.4 | 44.1 ± 14.8 | 0.09 |
| (mg/dL) | | | |
| Triglycerides (mg/dL) | | | 0.11 |
| Median | 147.0 | 159.0 | |
| Interquartile range | 107.0-205.5 | 122.0-223.0 | |
| Inflammatory variables | | | 0.03 |
| C-reactive protein | | | |
| (mg/L) | | | |
| Median | 3.8 | 4.7 | |
| Interquartile range | 1.9-8.9 | 2.2-18.0 | |
| Interieukin-6 (pg/mL) | | | 0.005 |
| Median | 9.9 | 12.8 | |
| Interquartile range | 5.6-17.4 | 7.6-25.3 | |
| sICAM-1 (ng/mL) | | | 0.08 |
| Median | 258.6 | 308.4 | |
| Interquartile range | 194.3-364.3 | 216.9-420.6 | |
| Metabolic variables | | | |
| Homocysteine (pmol/L) | | | <0.001 |
| Median | 13.5 | 15.2 | |
| Interquartile range | 11.1-16.4 | 12.6-20.4 | |
| Creatinine (mg/dL) | | | <0.001 |
| Median | 1.03 | 1.13 | |
| Interquartile range | 0.93-1.16 | 0.99-1.30 | |

Plus-minus values are means ±SD; LDL: low-density lipoprotein; HDL: high density lipoprotein; sICAM-I: soluble intercellular adhesion molecule 1. For normally distributed variables, P values were computed with t-tests; for skewed variables, P values were computed with the Wilcoxon rank-sum test for the difference in medians. The body-mass index is the weight in kilograms divided by the square of the height in meters. Revascularization refers to coronary-artery bypass surgery or percutaneous transluminal coronary angioplasty during follow-up. The left ventricular ejection fraction was available for 566 patients. To convert values for cholesterol to millimoles per liter, multiply by 0.02586; to convert values for triglycerides to millimoles per liter, multiply by 0.01129. To convert values for creatinine to micromoles per liter, multiply by 88.4.

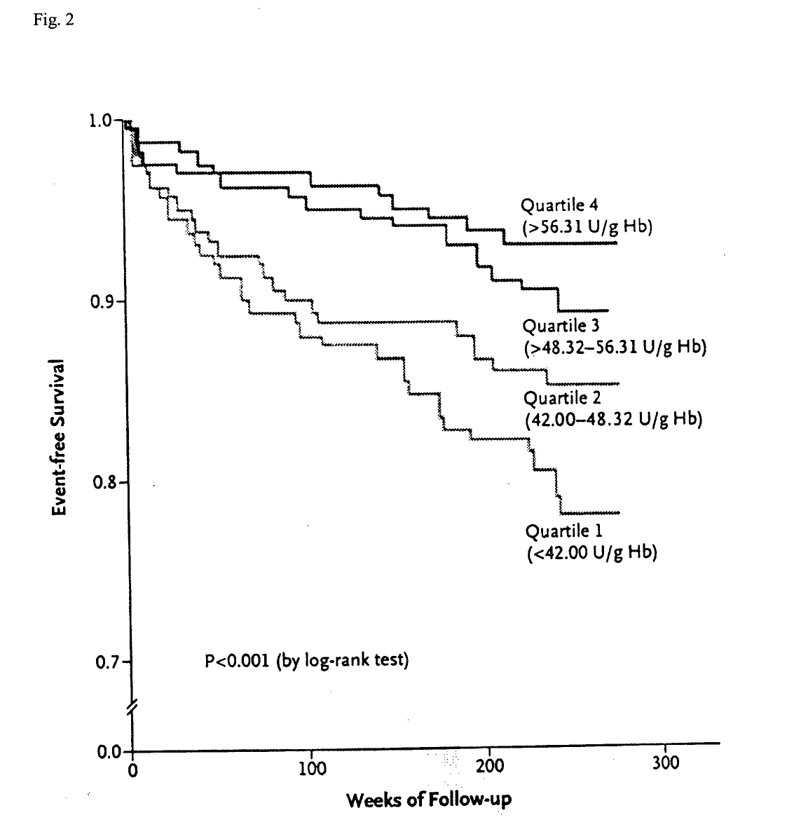

FIG. 2 shows the Kaplan-Meier curves for event-free survival according to quartile of GPx-1 activity. The unadjusted rate of cardiovascular events increased in a stepwise fashion across decreasing quartiles of base-line GPx-1 activity. The difference between the lowest and highest quartiles and the trend across all quartiles were significant (P<0.001 for both comparisons). The event rate for patients in the lowest quartile of GPx-1 activity (20.8 percent) was approximately three times that for patients in the highest quartile (7.0 percent). To place the effect in perspective, Table 2 presents the hazard ratios for cardiovascular events associated with an increase of 1 SD in various risk factors.

TABLE 2

| Variable | Hazard Ratio (95% CI) | P Value |
|---|---|---|
| Antioxidant enzymes and cofactors | | |
| GPx-1 | 0.69 (0.57-0.85) | <0.001 |
| Superoxide dismutase | 0.92 (0.72-1.19) | 0.54 |
| Selenium | 0.87 (0.68-1.11) | 0.26 |
| Lipid variables | | |
| LDL cholesterol | 1.06 (0.85-1.33) | 0.60 |
| HDL cholesterol | 0.76 (0.59-0.98) | 0.03 |
| Triglycerides | 1.17 (0.98-1.40) | 0.09 |
| Inflammatory variables | | |
| C-reactive protein | 1.18 (1.02-1.36) | 0.03 |
| Interieukin-6 | 1.15 (0.96-1.37) | 0.13 |
| sICAM-1 | 1.25 (0.99-1.58) | 0.06 |
| Metabolic variables | | |
| Homocysteine | 1.27 (1.11-1.44) | 0.001 |
| Creatinine | 1.23 (1.08-1.40) | 0.002 |

CI denotes “confidence interval.” The hazard ratio is the age- and sex-adjusted risk associated with an increase of 1 standard deviation in the variable.

The strongest predictors of the level of GPx-1 activity were smoking status and sex. Significantly lower levels of GPx-1 activity were observed in current smokers than in those who had never smoked (45.7 vs. 51.6 units per gram of hemoglobin, P<0.001). Former smokers also had lower levels of enzyme activity than those who had never smoked (48.2 vs. 51.6 units per gram of hemoglobin); however, this difference was not statistically significant. Furthermore, the level of GPx-1 activity was lower in men than in women (48.5 vs. 51.1 units per gram of hemoglobin, P=0.009). Women below 55 years of age had higher levels of GPx-1 activity than older women (54.1 vs. 50.5 units per gram of hemoglobin, P=0.12). No difference in GPx-1 activity was detected between patients with stable angina and those with unstable angina.

Although statin medication had no significant association with GPx-1 activity in the whole study population, the percentage of patients receiving statins was significantly higher among those in the highest quartile of GPx-1 activity than among those in the other three quartiles (30.6 percent vs. 19.5 percent, P=0.003). With this exception, no association was observed between the use of any cardiovascular medication and GPx-1 or superoxide dismutase activity. There was a weak but significant correlation of GPx-1 activity with homocysteine (r=−0.09, P=0.03) and selenium (r=0.09, P=0.04). Of all the inflammatory markers measured in this study, only soluble intercellular adhesion molecule 1 showed a moderate inverse correlation with GPx-1 activity (r=−0.11, P=0.02).

To assess the independent predictive value of GPx-1 activity, a series of Cox predictive models (Table 3) were employed. The inverse relation between GPx-1 activity and relative risk remained nearly unchanged after adjustment for cardiovascular risk factors and clinical features (model 2). Further adjustment for therapeutic variables as well as C-reactive protein (as a cluster representative of the inflammatory markers), homocysteine, and creatinine (model 3) also did not attenuate the relative risk associated with GPx-1 activity; patients in the highest quartile of GPx-1 activity had a hazard ratio of 0.29 (95 percent confidence interval, 0.14 to 0.60; P=0.001) as compared with those in the lowest quartile. Inclusion of interleukin-6 instead of C-reactive protein in the Cox predictive model had no effect on the hazard ratios. Similarly, in a subgroup of 566 patients in whom the ejection fraction had been measured, adjustment for this variable did not alter the hazard ratio associated with increasing quartiles of GPx-1 activity (data not shown).

TABLE 3

| | Quartile | | | | |
|---|---|---|---|---|---|
| | 1 | 2 | 3 | 4 | |
| | (<42.00 U/g | (42.00-48.32 U/g | (>48.32-56.31 U/g | (>56.31 U/g | P Value |
| Variable | hemoglobin) | hemoglobin) | hemoglobin) | hemoglobin) | for Trend |
| Total no. of | 159 | 159 | 161 | 157 | |
| patients | | | | | |
| Cardiovascular | 33 (20.8) | 23 (14.5) | 16 (9.9) | 11 (7.0) | <0.001 |
| events - n (%) | | | | | |
| Adjusted for | | | | | |
| age and sex | | | | | |
| (model 1) | | | | | |
| Hazard ratio | 1.0 | 0.65 | 0.42 | 0.29 | <0.001 |
| 95% CI | n/a | 0.38-1.11 | 0.23-0.77 | 0.15-0.58 | |
| P value | n/a | 0.13 | 0.005 | <0.001 | |
| Adjusted for | | | | | |
| risk factors | | | | | |
| (model 2) | | | | | |
| Hazard ratio | 1.0 | 0.71 | 0.49 | 0.32 | <0.001 |
| 95% CI | n/a | 0.41-1.21 | 0.26-0.90 | 0.16-0.65 | |
| P value | n/a | 0.20 | 0.02 | 0.001 | |
| Fully adjusted | | | | | |
| (model 3) | | | | | |
| Hazard ratio | 1.0 | 0.70 | 0.38 | 0.29 | <0.001 |
| 95% CI | n/a | 0.40-1.21 | 0.20-0.74 | 0.14-0.60 | |
| P value | n/a | 0.20 | 0.004 | 0.001 | |

In model 2, adjustment was made for body-mass index, presence or absence of hypertension, presence or absence of diabetes, smoking status, extent of vessel disease, presence or absence of ACS, and HDL (as a continuous variable). In model 3, further adjustment was made for vessel revascularization, statin and β-blocker therapy, homocysteine level, creatinine level, and CRP level (all as log-transformed continuous variables).

In previous reports from the entire AtheroGene cohort, the levels of several inflammatory markers, such as interleukin-18 and soluble vascular adhesion molecule 1, were found to be elevated among those at risk for subsequent coronary events. No statistically significant correlations were observed between GPx-1 activity and these two proteins. Inclusion of these variables in multivariate analyses did not attenuate the risk of future coronary events associated with decreased levels of GPx-1 activity.

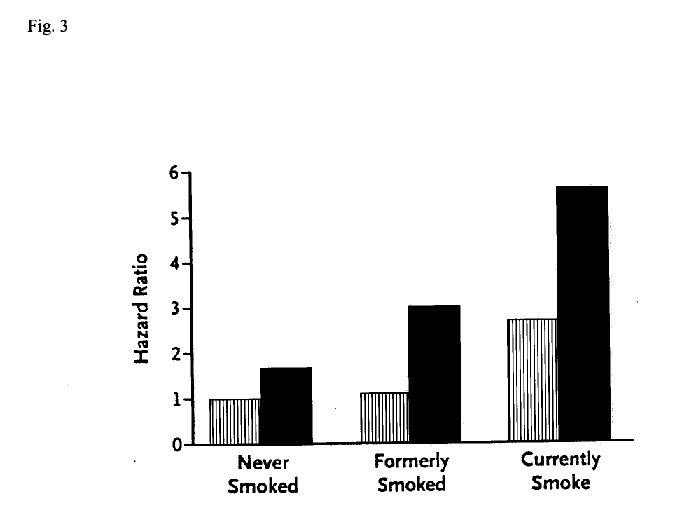

Because smoking status was associated with baseline GPx-1 activity as well as with coronary risk, the interaction between smoking status and GPx-1 activity was analyzed in more detail. The association between smoking and cardiovascular events was observed predominantly in patients with levels of GPx-1 activity below the median. As illustrated in FIG. 3, among those with low levels of GPx-1 activity (at or below the median value of 48.32 units per gram of hemoglobin), former smokers were at significant risk for cardiovascular events (hazard ratio, 3.0, as compared with patients who had never smoked and had high GPx-1 activity; 95 percent confidence interval, 1.5 to 5.9; P=0.001), as were current smokers (hazard ratio, 5.6; 95 percent confidence interval, 2.4 to 13.5; P<0.001). To a lesser extent, a significant increase in cardiovascular risk was also observed in former or current smokers in the subgroup of those with levels of GPx-1 activity above the median.

### Example 5

### Correlation of GPx-1 and Homocysteine Levels to Risk

Homocysteine has been reported to be a marker of risk in certain cardiac diseases. See, e.g., Nygard et al., N. Engl. J. Med. 337:230-6, 1997; Eikelboom et al., Ann. Intern. Med. 131:363-75, 1999; Alfthan et al., Atherosclerosis 106:9-19, 1994; Malinow, Circulation 81:2004-6, 1990. The following example explores the predictive value of homocysteine in relation to anti-oxidative GPx-1.

Baseline samples were obtained and GPx-1 from a prospective cohort of 605 consecutive patients with coronary artery disease for measurement of plasma homocysteine. Cardiovascular events (N=107) were registered during follow-up (mean: 7.1 years). Follow-up information was obtained about death from cardiovascular causes (n=81) and nonfatal myocardial infarction (n=26). Mean levels of variables in various subgroups were compared by Students's T-test for normal variables, Mann-Whitney-U test for skewed variables. The significance of differences in proportions was tested with the χ² statistic. In all survival analyses, the end points were death from cardiovascular causes and non-fatal myocardial infarction. To evaluate the combined effect of homocysteine and GPx-1 event plots according to median biomarker levels were estimated by the Kaplan-Meier method and compared with use of the log-rank test. The association of homocysteine and GPX-1 with outcome was evaluated in three Cox predictive models adjusted for potential confounders. In these analyses, skewed variables were log-transformed. P-values <0.05 were considered to be statistically significant.

Homocysteine levels were significantly elevated in the event group (15.4 (13.1/21.1) μmol/l vs. 13.4 (11.0/16.2) μmol/l; P<0.0001) in the overall study population, while GPx-1 concentrations were lower (45.3±13.1 U/g Hb vs. 50.2±11.0 U/g Hb; P<0.0001). In the subgroup with GPx-1 levels below median, homocysteine was independently predictive for adverse events. In the subgroup with high GPx-1 activity homocysteine lost its independent predictive value. This observation could be strengthened in the survival analysis of four subgroups divided according to their median levels of both biomarkers. The combination of low GPx-1 and elevated homocysteine revealed a 4.0-fold (P<0.0001) increased risk whereas the singular elevation of homocysteine did not go along with a significantly increased risk.

TABLE 4
Baseline characteristics of the overall study population
according to median (48.3 U/g Hb) GPx-1 baseline levels.

| | ≦48.3 U/g Hb | >48.3 U/g Hb | P |
|---|---|---|---|
| Variable | n = 302 | n = 303 | value |
| Age, years | 61.7 ± 9.8 | 61.7 ± 10.3 | 0.97 |
| Male sex (%) | 76.3 | 68.4 | 0.03 |
| Classical Risk Fac- | 26.5 ± 3.8 | 27.0 ± 3.3 | 0.28 |
| tors BMI, kg/m² | | | |
| History of diabetes | 17.8 | 13.5 | 0.15 |
| (%) | | | |
| History of hyper- | 66.1 | 71.4 | 0.16 |
| tension (%) | | | |
| Smokers (%) | 67.8 | 51.0 | <0.0001 |
| Hyperlipidemia | 54.9 | 63.5 | 0.03 |
| Homocysteine | | | |
| Metabolism | | | |
| Homocysteine (μmol/l) | 14.4 (11.5/17.9) | 13.5 (11.3/16.0) | 0.06 |
| Vitamin B₁₂ (pg/ml) | 390 (313/499) | 366 (300/476) | 0.08 |
| Folic Acid (ng/ml) | 7.0 (5.6/9.2) | 7.5 (5.6/9.4) | 0.39 |
| GPx-1 Metabolism | | | |
| Selenium (ng/ml) | 73.2 ± 33.2 | 77.5 ± 33.1 | 0.14 |
| Clinical Variables | | | |
| Multivessel Disease | 67.4 | 61.8 | 0.15 |
| (>2) (%) | | | |
| Left Ventricular | 61.9 ± 15.6 | 62.3 ± 15.6 | 0.78 |
| Ejection Fraction (%), | | | |
| (N = 539) | | | |
| Medication | | | |
| Statin Medication (%) | 24.7.5 | 28.6 | 0.55 |
| Betablocker (%) | 49.7 | 51.0 | 0.75 |
| Renal Function | | | |
| Creatinine, mg/dl | 1.1 ± 0.9 | 1.1 ± 0.3 | 0.47 |
| Inflammation | | | |
| CRP, (mg/l) | 3.9 (1.9/10.2) | 3.7 (1.9/9.2) | 0.79 |
| Fibrinogen, (mg/dl) | 331.0 ± 101.0 | 352.6 ± 121.6 | 0.75 |

Data presented are percentage of patients or means ± SD or median and 25^{th}/75^{th} interquartile range for skewed variables. Smoking status comprises current and former smokers
U/g Hb: Units per gram hemoglobin

Plasma concentrations of homocysteine are strongly and independently associated with the risk of future cardiovascular events only in CAD patients with low levels of anti-oxidative gluthatione peroxidase 1. High levels of GPx-1 seem to protect against adverse homocysteine effects.

TABLE 5
Hazard ratios and 95% confidence intervals (CI) for future cardiovascular
events according to quartiles of homocysteine plasma concentration

| | Quartile | | | | |
|---|---|---|---|---|---|
| | Q1 | Q2 | Q3 | Q4 | p-Value |
| Homocysteine (μmol/l) | <11.3 | 11.3-13.7 | 13.7-16.8 | >16.8 | |
| N = 605 | 149 | 152 | 153 | 151 | |
| % Events | 7.4 | 13.8 | 17.6 | 31.8 | |
| Model 1*, | | | | | |
| Hazard Ratio | 1 | 1.8 | 2.3 | 3.9 | <0.0001 |
| 95% CI | — | 0.9-3.7 | 1.1-4.6 | 2.0-7.6 | |
| P-Value | — | 0.12 | 0.02 | <0.0001 | |
| Model 2**, | | | | | |
| Hazard Ratio | 1 | 1.5 | 2.1 | 3.0 | 0.01 |
| 95% CI | — | 0.7-3.2 | 1.0-4.5 | 1.4-6.3 | |
| P-Value | — | 0.29 | 0.047 | 0.004 | |
| Model 3a^{†} | | | | | |
| GPx-1 below Median | N = 76 | N = 64 | N = 74 | N = 87 | |
| % Events | 7.9 | 17.2 | 28.4 | 36.8 | |
| Hazard Ratio | 1 | 3.0 | 5.57 | 5.31 | 0.045 |
| 95% CI | — | 0.59-14.83 | 1.22-25.52 | 1.13-24.92 | |
| P-Value | — | 0.19 | 0.027 | 0.03 | |
| Model 3b^{†}, | | | | | |
| GPx-1 above Median | N = 73 | N = 88 | N = 79 | N = 64 | |
| % Events | 6.8 | 11.4 | 7.6 | 25.0 | |
| Hazard Ratio | 1 | 0.31 | 0.70 | 0.88 | 0.40 |
| 95% CI | — | 0.07-1.44 | 0.22-2.30 | 0.26-3.0 | |
| P-Value | — | 0.14 | 0.56 | 0.84 | |

*controlled for age and sex
**additionally controlled for body mass index (squared), history of hypertension, diabetes, smoking status, HDL-cholesterol levels, multivessel disease, statin and betablocker therapy, and CRP, vitamin B12, folic acid and creatinine levels
^{†}subgroups divided according to median GPx-1 levels

A threshold effect was seen in patients with high homocysteine (above the median of 13.7 μmol/L) and low GPx-1 activity (below the median of 48.3 units/g Hb). These subjects revealed a 4.0-fold (95% CI 2.3-7.1; P<0.0001) increased risk of future cadiovascular events whereas neither patients with singularly elevated homocysteine nor subjects with singular GPx-1 activities below median seemed to be at significantly higher risk.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All such patents and publications are herein incorporated by reference to the same extent as if each individual publication was specifically and individually indicated to be incorporated by reference. Also incorporated by reference in this the figures in U.S. Application Ser. No. 60/602,997.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

We claim:

1. A method of assigning a risk of one or more future clinical outcomes to a subject, the method comprising:
determining the presence, amount, or enzymatic activity of glutathione peroxidase-1 (“GPx-1”) in a sample obtained from said patient; and
correlating the presence, amount, or enzymatic activity of said GPx-1 to said risk of one or more clinical outcomes for the subject.

2. A method according to claim 1, wherein said subject is suffering from a cardiovascular condition.

3. A method according to claim 1, wherein said subject is clinically normal with regard to cardiovascular conditions.

4. A method according to claim 1, wherein said one or more clinical outcomes are selected from the group consisting of death, stroke, myocardial infarction, rehospitalization, coronary revascularization, and congestive heart failure.

5. A method according to claim 2, wherein said cardiovascular condition is selected from the group consisting of acute coronary syndrome, atherosclerosis, ischemic stroke, intracerebral hemorrhage, subarachnoid hemorrhage, transient ischemic attack, systolic dysfunction, diastolic dysfunction, aneurysm, aortic dissection, myocardial ischemia, angina pectoris, myocardial infarction, congestive heart failure, dilated congestive cardiomyopathy, hypertrophic cardiomyopathy, restrictive cardiomyopathy, cor pulmonale, arrhythmia, valvular heart disease, endocarditis, pulmonary embolism, venous thrombosis, and peripheral vascular disease.

6. A method according to claim 2, wherein said cardiovascular condition is an acute coronary syndrome.

7. A method according to claim 1, wherein said correlating step comprises comparing a GPx-1 enzymatic activity measured in said sample to a threshold GPx-1 enzymatic activity level, whereby, when said GPx-1 enzymatic activity measured in said sample is less that said threshold BNP level, said patient is predisposed to one or more of said clinical outcomes.

8. A method according to claim 1, wherein the correlating step comprises a GPx-1 enzymatic activity measured in said sample to a threshold GPx-1 enzymatic activity level, wherein a GPx-1 enzymatic activity measured in said sample less than said threshold GPx-1 enzymatic activity level is indicative of a first clinical outcome and a GPx-1 enzymatic activity measured in said sample greater than said threshold GPx-1 enzymatic activity level is indicative of a second clinical outcome.

9. A method according to claim 8, wherein said threshold GPx-1 enzymatic activity level provides an odds ratio of about 4 or greater or about 0.25 or less.

10. A method according to claim 8, wherein said threshold GPx-1 enzymatic activity level provides a hazard ratio of about 1.25 or greater or about 0.8 or less.

11. A method according to claim 8, wherein said threshold GPx-1 enzymatic activity level is greater than a median GPx-1 enzymatic activity level measured in samples from subjects suffering from an acute coronary syndrome.

12. A method according to claim 8, wherein wherein said threshold GPx-1 enzymatic activity level is less than about 48 U/g hemoglobin.

13. A method according to claim 1, further comprising determining the presence or amount of one or more other subject-derived markers in said sample, and said correlating step comprises correlating the presence, amount, or enzymatic activity of said GPx-1 and said one or more other subject-derived markers to said risk of one or more clinical outcomes for the subject.

14. A method according to claim 13, wherein said other subject-derived markers comprise one or more markers independently selected from the group consisting of markers related to myocardial injury, markers related to blood pressure regulation, markers related to coagulation and hemostasis, markers related to inflammation, and markers related to apoptosis.

15. A method according to claim 14, wherein said one or more other subject derived markers comprise one or more markers related to myocardial injury.

16. A method according to claim 14, wherein said one or more other subject-derived markers comprise one or more markers related to blood pressure regulation.

17. A method according to claim 14, wherein said one or more other subject-derived markers comprise one or more markers related to coagulation and hemostasis.

18. A method according to claim 14, wherein said one or more other subject-derived markers comprise one or more markers related to apoptosis.

19. A method according to claim 13, wherein said other subject-derived markers comprise one or more markers selected from the group consisting of caspase-3, thrombus precursor protein, creatine kinase-MB, total cardiac troponin I and/or T, free cardiac troponin I and/or T, complexed cardiac troponin I and/or T, myoglobin, B-type natriuretic peptide, NT-proBNP, homocysteine, C-reactive protein, D-dimer, myeloperoxidase, and markers related thereto.

20. A method according to claim 19, wherein said other subject-derived markers comprise BNP or a marker related thereto.

21. A method according to claim 19, wherein said other subject-derived markers comprise free cardiac troponin I, complexed cardiac troponin I, free and complexed cardiac troponin I, free cardiac troponin T, complexed cardiac troponin T, free and complexed cardiac troponin T, or a marker related thereto.

22. A method according to claim 19, wherein said other subject-derived markers comprise C-reactive protein or a marker related thereto.

23. A method according to claim 19, wherein said other subject-derived markers comprise caspase-3 or a marker related thereto.

24. A method according to claim 19, wherein said other subject-derived markers comprise myeloperoxidase or a marker related thereto.

25. A method according to claim 19, wherein said other subject-derived markers comprise homocysteine.

26. A method according to claim 1, wherein the sample is from a human.

27. A method according to claim 1, wherein the sample is selected from the group consisting of blood, serum, and plasma.

28. A method according to claim 1, wherein said GPx-1 is erythrocyte GPx-1.
